(19) United States
(12) Patent Application Publication
(10) Pub. No.: US 2003/0211022 A1
(43) Pub. Date: Nov. 13, 2003

(54) Method and apparatus for decontaminating water or air by a photolytic and photocatalytic reaction

(76) Inventors: Karl J. Gross, Fremont, CA (US); Arlyn Antolak, Patterson, CA (US)

Correspondence Address: Timothy Evans, MS 9031, Sandia National Laboratories, 7011 East Avenue, Livermore, CA, 94550, US

(21) Appl. No.: 10/143,310

(22) Filed: May 10, 2002

(51) Int. Cl. B01D 11/04; B01D 11/02; A61L 2/00

(52) U.S. Cl. 422/292; 422/256; 422/261; 422/307

(57) ABSTRACT

Photolytic and photo-catalytic reactions have the potential to passivate water- or air-borne bio-hazardous materials. This invention describes a device to be used as a means for disinfecting water contaminated with organic compounds or biological agents such as bacteria, or viruses. The present invention relates to a device utilizing an inert substrate matrix to support a photoactive catalyst and a means for transmitting high energy light, especially ultraviolet light. The matrix presents a large surface area in direct contact with the contaminated water or air. The matrix transmits or is transparent to light emanating from a source such as a UV lamp. The substrate matrix provides a means for light to interact in close proximity with the photoactive catalysts and organic matter in the water or air. The photoactivated catalyst accelerates the decomposition of biological matter in the water or air, effectively disinfecting the water or air as it comes into contact with the photoactivated agent held on the substrate.

### STATEMENT OF GOVERNMENT INTEREST

This invention was made with Government support under government contract no. DE-AC04-94AL85000 awarded by the U.S. Department of Energy to Sandia Corporation. The Government has certain rights in the invention, including a paid-up license and the right, in limited circumstances, to require the owner of any patent issuing in this invention to license others on reasonable terms.

### BACKGROUND OF THE INVENTION

1. Field of the Invention

The present invention relates generally to treatment of air or water via both photolytic and photo-catalytic reactions initiated using high energy light, particularly ultraviolet (UV) light. More specifically, the subject invention relates to a novel apparatus utilizing a substrate supported photo-catalytic material and methods to enhance photo-catalytic reactivity of said materials.

Ultraviolet (UV) reaction chambers are typically employed in the ultra-purification of water as well as in the conditioning of other fluids generally. Such sanitization processes typically entail microbial destruction, and total organic content (TOC) reduction. In the absence of a catalyst, these reactions are commonly referred to as photolytic reactions while if carried out in the presence of a catalyst, these reactions are known as photo-catalytic reactions.

Photo-catalysis belongs to the family of Advanced Oxidation Processes (AOP) that utilize an oxidant species to break the chemical bonds between carbon atoms and other atoms of carbon, nitrogen, chlorine, sulfur, fluorine, and other elements. The array of species that have been affected by photo-catalysis in laboratory studies include, inter alia, simple organic compounds, chlorinated organic compounds, AOPs include hydrogen peroxide and a metal in the presence of ultraviolet (UV) light to promote the production of hydroxyl radicals. This combination is commonly referred to as Fenton's Reagent.

Photo-catalysis is an AOP, based on a solid semiconductor material that is bombarded with UV radiation to excite the electrons and holes within the semiconductor material to produce oxidation-reduction (redox) reactions. Photo-catalytic reactions are heterogeneous or homogenous chemical reactions that take place on semiconductor surfaces in the presence of an energy source sufficient to overcome the energy gap of the semiconductor material to promote electron and hole mobility within the valence and conductance bands of the semiconductor material. Classical reactions take place in aqueous solutions where the semiconductor material produces hydroxyl and peroxide species to oxidize organic compounds to carbon dioxide, water, and inorganic acids.

2. Prior Art

The art is often described in terms of either a suspended/slurried photocatalyst or a fixed photocatalyst. Suspended catalysts are those utilizing fine particles of a semiconductor material, generally to increase catalyst surface area. U.S. Pat. No. 5,589,678 (Butters, et al.) provides a description of photo-catalytic slurries. Suspended catalysts are limited to maximum concentrations in the fluid since they (1) increase turbidity, (2) absorb light, and (3) refract light, and thus decreasing overall UV transmission in an illuminated reactor.

Fixed catalysts, to which the subject invention are directed, employ a singular or multi-pieced support or substrate to which the photocatalyst is applied. Fixed catalysts have been perceived as having less overall catalyst surface area then suspended catalysts, but do not require removal and recovery of the suspended catalyst particles. An example of a fixed catalyst support design is presented in U.S. Pat. No. 5,790,934 (Say, et al.). The Say invention utilizes multiple fins located in a radial or longitudinal arrangement and suffers from various shortcomings and limitations. First, the fixed substrate fins are situate at a certain distance away from the UV source. Reactivity is greatest in close proximity to the light source and decreases with distance.

Some fixed catalyst substrates have been proposed to increase overall catalyst surface area through catalyst absorption onto silica gel, zeolites, carbon black, and porous metals, however, the micropores of these fixed catalysts may not allow sufficient illumination to penetrate for efficient catalyst activation. Also, these materials are packed into a reactor where proper illumination of some surfaces of a majority of the catalysts may not be accomplished.

U.S. Pat. No. 5,501,801 (Zhang, et al.) illustrates the use of silica gel and zeolite substrates as photo-catalytic supports.

Another fixed substrate design is the use of titanium metal pieces (rods, spheres, beads, chunks, and the like) that are oxidized to form the desired titanium dioxide layer. As discussed in U.S. Pat. No. 5,868,924 (Nachtman, et al.) and U.S. Pat. No. 5,395,552 (Melanson, et al.), titanium metal, or its alloys, are inserted into a UV chamber along the length of the UV source, at a distance away from the UV source.

Based on the above prior art, there has clearly been demonstrated an effort to enhance photo-catalysis through, among other things, development of novel fixed-catalyst substrates. As will become apparent upon review of the detailed description below, Applicant has developed a new and improved fixed-catalyst substrate with several advantages heretofore unobserved.

### SUMMARY OF THE INVENTION

A variety of photocatalytic reactions involving different Transition metal oxides have been used to decompose organic contaminants, particularly oxides of those metals listed in New IUPAC Groups 4-12 of the Periodic Table of Elements, most particularly TiO₂, ZnO, Fe₂O₃, and WO₃. Also used are as some semiconductor materials (CdS). In particular, it has been shown that TiO₂ exhibits one of the highest photocatalytic activities to efficiently kill viruses, bacteria, fungi, and algae for water and air purification. When such materials are illuminated with (near-UV) light having energy greater than the band gap of the incident material, electrons in the valance band are excited to the conduction band, creating electron-hole (e-h) pairs. Such photo-generated holes have strong oxidation power and easily react with oxygen to produce a number of highly reactive species, such as hydroxyl free radicals (OH⁻ and HO₂⁻) and superoxide ions (O₂⁻). These latter species readily decompose organic compounds leading to cellular membrane destruction and genetic molecular damage. Finally, there is evidence to suggest that smaller (≦100 nanometers) photoactive particles cause more rapid intracellular damage and that the disinfecting activity continues even after the UV exposure is terminated.

Standard activated carbon filtration typically removes chemical but not biohazards from drinking water. The use of UV light and catalysts has been investigated for large-scale water purification, such as at a water treatment facility. However, there are some practical limitations to such a large-scale facility, and these operations do not circumvent contamination that may occur between the facility and the end-user.

What is needed is a single-user water disinfecting device using UV light for disinfecting water.

It is therefore an object of this invention to provide a device to purify and/or disinfect tap water by using UV light, a catalyst, and a means for holding the catalyst, and a means for exposing the water to both the UV light and the catalyst.

Since the key to the success of a single-user water purification device is to increase the overall efficiency of the device, it is another object of this invention to provide a method for increasing the speed at which the disinfection process proceeds.

Yet another object of this invention is to provide a device utilizing a mixed oxide catalyst.

Still another object of the invention is to provide a device utilizing a nanometer-sized photocatalytic TiO₂ particles or other nanometer-sized mixed oxide photocatalysts.

Another object of this invention is to provide a method for applying the metal oxide photocatalysts to a support substrate.

Another object of the invention is to provide a means for efficiently distributing UV light to the nanometer-sized metal oxide catalysts.

Still another object of this invention is to provide UV transparent fiber optic bundles, pyrolyzed foams and gels, beads or particles for distributing UV light to the mixed oxide catalysts.

An additional object of the invention is to provide an accelerated, dual disinfecting action by combining photolytic (UV) and photocatalytic (UV+photocatalyst) reactions.

It shall also be an object of this invention to provide an apparatus and an associated method for purifying ambient air used for building or vehicular ventilation.

These and further objects, features and advantages of the invention will become apparent to those having skill in these arts from the following detailed description of the invention when taken together with the accompanying drawings and appended claims.

### BRIEF DESCRIPTION OF THE DRAWINGS

FIG. 1 illustrates a first embodiment of the invention comprising a plurality of catalyst coated optical fibers attached to a UV light source and immersed in a quantity of water to be purified.

FIG. 2 illustrates a second embodiment of the invention comprising a source of UV light surrounding a substrate, that is transparent to UV light, on which a catalyst material is supported.

### DETAILED DESCRIPTION OF THE INVENTION

The present invention comprises a reactor utilizing an inert substrate matrix to support photoactive catalysts and a means for illuminating the photoactive catalysts with a source of ultraviolet light. The substrate matrix may be comprised of one or a combination of the same or different UV transparent materials, or the substrate matrix may comprise a plurality of the same or different UV transparent materials, where the materials may be any of optical fibers, a pyrolyzed porous foam, a gel, a sol-gel or an aerogel, glass or plastic wool, a woven fabric, and glass or plastic beads or particles made of a material capable of transmitting or propagating UV light. In addition, the reactor may contain an activated carbon portion for final filtering

Effective photocatalysts comprise, among other materials, one or more metal oxides. In particular, the present invention utilizes finely divided titanium oxide (TiO₂) as the catalyst. The photoactivity of these catalysts is enhanced by reducing the mean diameters of these oxide powders below about 100 nm in size. It has been shown that these nano-powders can be produced by well-known processes such as flame spray pyrolysis, sol-gel, and surfactant-based supramolecular self-assembly techniques. Furthermore, flame spray pyrolysis may allow the direct deposition of the catalysts onto the surface of the matrix. It is also known that oxide mixture which incorporate aluminum oxide are much more dispersible in water or organic solutions making them amenable to coating surfaces by wetting and evaporation. Finally, commonly owned U.S. Pat. No. 6,188,812, herein incorporated by reference, describes a process for coating optical fibers with thin layers of a sol-gel.

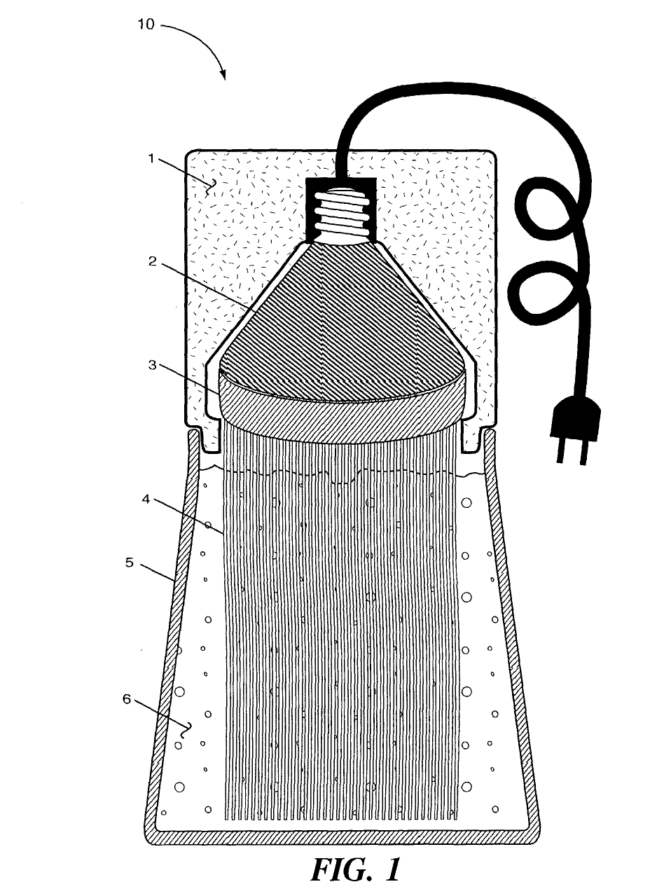

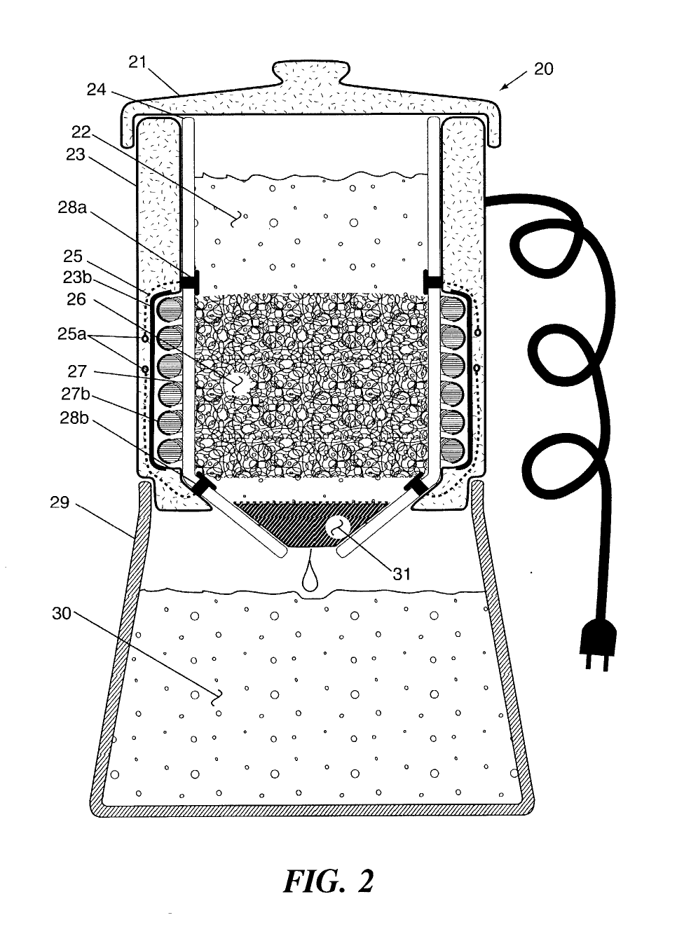

The device of the present invention is best described and illustrated with reference to FIGS. 1 and 2 which show two embodiments similar to the small activated-carbon filtration systems used in homes today to reduce dissolved trace chemicals and bacterial agents.

FIG. 1 shows such a simple first embodiment of a filtration device 10 based on a screen or “apron bundle” 4 of optical fibers that act as both a photoactive substrate and as a conduit for transmitting light radiation at wavelengths below about 550 nm, and preferably in the range of about 200 nm to about 400 nm. Light from lamp 2 at the top of the filtration device 10 is passed into light collection means 3 where the light is then conducted into and through optical fiber apron 4. Each of the fibers comprising apron bundle 4 include a coating comprising TiO₂ or a combination of photocatalytic metal oxides applied by gel coating, flame-spray pyrolysis or vapor deposition. The assembly of lamp 2, light collection means 3, and apron bundle 4 are held in downward position within container lid 1 which sized to fit over water container 5. Bacteria and other biological matter contained in contaminated water 6 circulating around and through the apron of fibers would be killed by both UV photolytic reactions and by enhanced photocatalytic decomposition as light from lamp 2 leaks through the walls of the fibers onto the photoactive catalysts. Harmful organic contaminants would also be decomposed into benign intermediates and by-products.

In a second embodiment of this invention shown in FIG. 2, water purification filter device 20 operates in a batch mode much like a drip coffee maker. The assembly comprises a container 23 providing structural support for the assembly, an interior open-ended tube 24 contained within container 23 that holds the contaminated water 22 and a substrate matrix 26 coated with a photoactive catalyst, a high energy light source 27 housed within container 23, a receptacle, or basin 29, for collecting purified water 30, and a lid 21 for covering the surface of the assembly.

The substrate matrix 26 used to support the photo-catalyst is fabricated from an expanded open material selected from the group of glass or plastic wool, porous pyrolyzed foams, glass or plastic beads, or any other generally inert particles made of a material capable of transmitting or propagating light, particularly UV light. As before, the substrate is coated with nanometer-sized photocatalyic materials. Coating is performed by known methods such as by gel coating, flame-spray pyrolysis or vapor deposition. Matrix 26 is placed in the bottom half of an interior tube 24 contained within open ended vessel 23 and contaminated water 22 is added at the top of container 23 so that it covers and passes through substrate matrix 26 and is collected in a basin 30 located below vessel 23. Water flow out from interior tube 24 at its lower end but it's flow restricted by a reduction in the opening at this lower end and by a water-permeable plug or assembly 31 comprising a quantity of activated carbon/charcoal which blocks the opening and acts as a final filter.

Interior tube 24 is designed to be transparent to visible/UV light radiation and is itself surrounded by a high intensity lamp or light source 27 in the form of a coiled tube that supplies high energy light at wavelengths below about 550 nm, and preferably in the range of about 200 nm to about 400 nm, to activate the photo-catalyst. Additionally, lamp or light source 27 coiled tube includes reflection means 27b along the half of the coiled tube exterior surface distal to interior tube 24, wherein reflection means 27b is for redirecting light propagating away from the assembly interior back toward interior tube 24. (Alternatively, interior surface of open ended vessel 23 adjacent to lamp 27 may be coated with a reflecting means 23b for redirecting light propagating toward that surface.)

Interior tube 24 also includes upper and lower sets of electrodes 28a and 28b for sensing the presence of water contained in the tube. Electrodes 28a and 28 are positioned along the surface of the inside wall of the interior tube 24 just above and below the level of substrate matrix 26 and communicate with light source 27 through electrical wires 25 and connections 25a penetrating the tube wall. Uppermost electrodes 28a turn lamp 27 on when water is present and lowermost electrode(s) 28b turn lamp 27 off when water is absent. A third set of electrodes (not shown) placed a distance above upper electrode 24, and working together with 24 electrode, can also serve as a control assembly for opening and closing a valve to replenish water in container 23.

Assembly 20, therefore, is designed so that water flows by gravity through substrate matrix 26 where high energy visible/UV light from external tube or lamp 27 is transmitted through the matrix to the photocatalyst which is in contact with contaminated water 22. Bacteria and other biological matter in the water are killed through catalytically enhanced decomposition at the sites of the photoactive catalysts.

Finally, while embodiments of water filtration devices, together with materials, processes, device configurations, etc., have been described and/or illustrated to exemplify and teach the principles of the invention, such are not intended to be limiting. Modifications and changes may become apparent to those skilled in the art, and it is intended that the invention be limited only by the scope of the appended claims. In particular, it would be apparent to those skilled in the art that the embodiment shown in FIG. 2 if combined with an air inlet fan could be reconfigured to provide a device for filtering household or workspace air. By removing lid 21 and basin 29 air may be passed through the filter rather than water. Additional stages comprising the lamp and substrate portion of the device ganged together in series would probably be necessary in order to overcome the relatively low residency time of the air passing through any one layer.

What is claimed is:

1. A filtration device for decontaminating a fluid, comprising:
a container for holding a quantity of a fluid contaminated with organic or biological matter;
one or more metal oxide photoactive catalysts;
means for supporting said metal oxide catalyst in said contaminated fluid;
a high energy light source, said light source for providing high energy light having a frequency or wavelength capable of initiating a photo-catalytic reaction at a surface of said photoactive catalyst; and
means for directing said high energy light onto said surface of said photoactive catalyst.

2. The filtration device of claim 1, wherein said metal oxide catalyst comprises a plurality of particles having average diameters below about 100 nm.

3. The filtration device of claim 1, wherein said one or more metal oxide photoactive catalysts comprises oxides of titanium and at least one additional metal oxide catalyst selected from the group consisting of the Transition metal oxides listed in New IUPAC Groups 4-12 of the Periodic Table of Elements.

4. The filtration device of claim 1, wherein said metal oxide catalyst consists essentially of TiO₂.

5. The method for applying said metal oxide catalyst to the support means of claim 1, comprising the steps of:
dispersing said metal oxide catalyst into an aqueous or an organic solution; and
coating said support means with some of said aqueous or an organic solution.

6. The method of claim 5, wherein said aqueous or an organic solution includes finely divided Al₂O₃ particles, wherein said Al₂O₃ particles are added to aid in dispersing said TiO₂.

7. The applying said metal oxide catalyst to the support means of claim 5, further including a process selected from the group consisting of flame spray pyrolysis, vapor deposition, sol-gel coating, and surfactant-based supramolecular self-assembly coating.

8. The filtration device of claim 1, wherein said means for supporting said metal oxide catalyst comprises a material capable of transmitting or propagating UV light.

9. The filtration device of claim 8, wherein said material capable of transmitting or propagating UV light comprises a material selected from the group consisting of glass wool, plastic wool, glass beads, plastic beads, and porous pyrolyzed foams.

10. The filtration device of claim 1, wherein said high energy light source comprises a lamp.

11. The filtration device of claim 10, wherein said lamp providing light having a wavelength below about 550 nm.

12. The filtration device of claim 10, wherein said lamp providing light having a wavelength below about 400 nm.

13. The filtration device of claim 1, wherein said means for directing said high energy light comprises mirror surface for focusing said light into a material capable of transmitting or propagating said light.

14. The filtration device of claim 13, wherein said means for directing said high energy light comprises a plurality of optical fibers.

15. The filtration device of claim 1, further including a final filter comprising a quantity of activated carbon or charcoal.

16. The filtration device of claim 10, wherein said fluid is water.

17. The filtration device of claim 16, further including means for turning said lamp on if water is present and means for turning said lamp off if water is not present.

18. The filtration device of claim 10, wherein said fluid is air.

19. The filtration device of claim 18, further including means for drawing air across said metal oxide catalyst.
